(19) United States
(12) Patent Application Publication
(10) Pub. No.: US 2025/0380932 A1
(43) Pub. Date: Dec. 18, 2025

(54) Packaging For Biological Samples

(71) Applicant: Iovance Biotherapeutics, Inc., San Carlos, CA (US)

(72) Inventors: Nicholas T. Rojeski, Maplewood, NJ (US); Jayson Amador, Fairfield, CA (US); Matthew Parker, Ardmore, TN (US)

(21) Appl. No.: 18/878,574

(22) PCT Filed: Jun. 29, 2023
(86) PCT No.: PCT/US2023/069323

§ 371 Date: Dec. 23, 2024

Related U.S. Application Data
(60) Provisional application No. 63/356,788, filed on Jun. 29, 2022.

(51) Int. Cl. A61B 10/00; A61B 90/94; B65B 5/04; B65B 25/00; B65B 61/18; B65B 61/26; B65D 25/10; B65D 77/04; B65D 81/18; B65D 81/26; F25D 11/00; G01K 1/14

(52) U.S. Cl. CPC A61B 10/0096; A61B 90/94; B65B 5/045; B65B 25/00; B65B 61/182; B65B 61/26; B65D 25/10; B65D 77/04; B65D 81/18; B65D 81/264; F25D 11/003; G01K 1/14; B65D 2203/02; B65D 2401/05; B65D 2577/042

(57) ABSTRACT

A packaging and transporting device for a biological sample including an insert, a securing strip, a securing element, a temperature sensor, a plurality of labels, a shipping pouch sized and shaped to receive a shipping document, and a transport bag configured to receive a sample container. The insert comprises a plurality of cutouts sized and shaped to receive the securing strip, the securing element, and the temperature sensor. At least one of the plurality of labels includes patient information.

### CROSS REFERENCE TO RELATED APPLICATIONS

This application claims the benefit of U.S. Provisional Patent Application No. 63/356,788 filed Jun. 29, 2022 entitled “Packaging For Biological Samples”, the content of which is incorporated by reference herein in its entirety.

### TECHNICAL FIELD

The present disclosure generally relates to packaging for biological samples, and more particularly, packaging for securing biological samples for shipping and transportation.

### SUMMARY

One embodiment of the present disclosure provides a packaging and transporting device for a biological sample, the packaging and transporting device includes an insert, a securing strip, a securing element, a temperature sensor, the insert having a plurality of cut outs sized and shaped to receive the securing strip, the securing element, and the temperature sensor, a plurality of labels, a shipping pouch sized and shaped to receive a shipping document, and a transport bag configured to receive a sample container.

In some embodiments, the plurality of cut outs include a first cut out, a second cut out, and a third cut out. The first cut out of the insert may be configured to receive and store the securing strips, the second cut out of the insert may be configured to receive and store the temperature sensor, and the third cut out of the insert may be configured to receive and store the securing element.

In some embodiments, the insert is disposed between the transport bag and the plurality of labels. The insert may be disposed between the transport bag and the plurality of labels, and the transport bag may be disposed between the shipping pouch and the insert.

In some embodiments, an inner shipping container sized and shaped to receive the transport bag and a cooling engine, wherein the inner shipping container is smaller in size than the packaging and transporting device. The plurality of labels may include a first label configured to be applied to the sample container and a second label configured to be applied to the inner shipping container. The securing strips may be configured to leave a mark on the inner shipping container if removed or altered.

In some embodiments, the insert includes three or more securing strips. The three or more securing strips may be disposed within the plurality of cut outs of the insert.

In some embodiments, the securing strip is disposed within one of the plurality of cut outs of the insert. The insert may include four or more securing elements. The four or more securing elements may be disposed within the plurality of cut outs of the insert.

In some embodiments, the patient information comprises identifying patient information. The identifying patient information may comprise a patient's name, birthdate, age, etc.

In some embodiments, the packaging and transporting device further includes a cooling engine configured to maintain a temperature range of approximately −80° C. to approximately 10° C. upon activation of the cooling engine.

In some embodiments, the transport bag includes an absorbent sheet configured to absorb leakage of the sample container. The transport bag may be configured to withstand up to 95 kPa without leaking.

In some embodiments, the packaging and transporting device includes two or more labels.

In some embodiments, the temperature sensor is disposed within one of the plurality of cut outs of the insert. The insert may be disposed between the plurality of labels and one of the shipping pouch and the transport bag.

In some embodiments, the patient information comprises de-identified patient information.

In some embodiments, the packaging and transporting further includes a kit container housing the insert, the plurality of labels, the shipping pouch, and the transport bag.

Another embodiment of the present disclosure provides a packaging and transporting device for a biological sample, the device includes a packaging kit having an insert having a securing strip, a securing element, and a temperature sensor, wherein the insert includes a plurality of cut outs sized and shaped to receive the securing strip, the securing element, and the temperature sensor, a plurality of labels including one or more of patient information and destination information, a transport bag configured to receive a sample container containing the biological sample, a shipping pouch, a cooling engine configured to maintain a temperature range of approximately 0° C. to approximately 10° C., an inner shipping container, and an outer shipping container sized and shaped to receive the inner shipping container, the cooling engine, and the packaging kit. In some embodiments, the inner shipping container includes an area to receive at least one of the plurality of labels and the inner shipping container is sized and shaped to receive the cooling engine and the transport bag.

In some embodiments, the insert is disposed between the transport bag and the plurality of labels. The transport bag may be disposed between the shipping pouch and the insert.

Another embodiment of the present disclosure provides a method for packaging a biological sample, the method including providing a packaging kit having a transport bag and an insert storing a securing element, a securing strip, and a temperature sensor, securing a sample container with the securing element, wherein the sample container includes the biological sample, applying a first label to the secured sample container, the first label including at least one of patient information, inserting the labeled sample container into the transport bag, placing the transport bag into an inner shipping container, wherein the inner shipping container comprises a cooling engine and the temperature sensor, closing the inner shipping container such that the transport bag, the cooling engine, and the temperature sensor are disposed within the inner shipping container, securing the inner shipping container in a closed position using the securing strip such that attempted access to an interior of the inner shipping container is evident, and applying a second label to the inner shipping container, the second label including at least one of patient information and destination information.

In some embodiments, the packaging kit further includes a shipping pouch and a plurality of labels including the first label and the second label. Within the packaging kit, the insert may be disposed between the transport bag and the plurality of labels. Within the packaging kit, the transport bag may be disposed between the shipping pouch and the insert.

In some embodiments, the method further includes removing the securing strip from a first cut out of the insert, removing the temperature sensor from a second cut out of the insert, and removing the securing element from a third cut out of the insert.

In some embodiments, the first cut out of the insert is configured to receive and store the securing strip. The second cut out of the insert may be configured to receive and store the temperature sensor. The third cut out of the insert may be configured to receive and store the securing element.

In some embodiments, the method further includes activating the cooling engine such that the cooling engine is configured to keep the inner shipping container within a predetermined temperature range when the activated cooling engine is disposed within the inner shipping container. The predetermined temperature range may be 0° C. to 10° C.

In some embodiments, the method further includes transporting the inner shipping container having the second label to a destination.

In some embodiments, the method further includes retrieving the biological sample from a patient.

In some embodiments, the method further includes combining the biological sample with a transport media, wherein the transport media is configured to prevent degradation of the biological sample. The biological sample may comprise a plurality of tumor tissue retrieved from a patient.

In some embodiments, the method further includes the transport bag includes an absorbent configured to absorb leakage of the sample container.

In some embodiments, the securing strip is configured to leave a mark on the inner shipping container if removed or altered.

In some embodiments, the packaging kit includes three or more securing strips. The packaging kit may include four or more securing elements. The packaging kit may include two or more labels.

In some embodiments, the transport bag is configured to withstand up to 95 kPa without leaking.

In some embodiments, the patient information is de-identified patient information. The patient information may comprise identifying patient information. The patient information may comprise the patient's name, birthdate, and/or age.

In some embodiments, the packaging kit includes a kit container, the kit container configured to receive and store the transport bag and the insert.

Another embodiment of the present disclosure provides a method for packaging a biological sample, the method including receiving an outer shipping container having a cooling engine, an inner shipping container, and a packaging kit, wherein the packaging kit having a transport bag, a shipping pouch, a plurality of labels and an insert storing a securing element, a securing strip, and a temperature sensor, removing from the outer shipping container the cooling engine, the inner shipping container, and the packaging kit, removing from the packaging kit the transport bag, the shipping pouch, the plurality of labels, and the insert, wherein the insert includes cut outs sized and shaped to receive the securing element, the securing strip, and the temperature sensor, removing from the insert the securing element, the securing strip, and the temperature sensor, placing the biological sample within a sample container, the biological sample including a tumor tissue and a transport media, securing the sample container with the securing element, applying a first label of the plurality of labels to the secured sample container, the first label including at least one of patient information and destination information, inserting the labeled sample container into the transport bag, the transport bag including an absorbent sheet configured to absorb leakage of the labeled sample container within the transport bag, activating the cooling engine such that the cooling engine is configured to keep the inner shipping container within a predetermined temperature range when the activated cooling engine is disposed within the inner shipping container, placing the transport bag, the cooling engine and the temperature sensor into the inner shipping container, closing the inner shipping container such that the transport bag, the cooling engine, and the temperature sensor are disposed within an interior of the inner shipping container, securing the inner shipping container in a closed position using the securing strip such that access to an interior of the inner shipping container is prevented, wherein the securing strip is configured to leave a mark or residue on the inner shipping container if removed or altered, and applying a second label of the plurality of labels to the inner shipping container, the second label including at least one of patient information and destination information.

### BRIEF DESCRIPTION OF THE DRAWINGS

The following detailed description of embodiments of the packaging for biological samples will be better understood when read in conjunction with the appended drawings of an exemplary embodiment. It should be understood, however, that the disclosure is not limited to the precise arrangements and instrumentalities shown.

FIG. 1 is a front perspective view of a package container in accordance with an exemplary embodiment of the present disclosure;

FIG. 2 is a top perspective view of a cooling block in accordance with an exemplary embodiment of the present disclosure;

FIG. 3 is a top perspective view of an inner shipping container in accordance with an exemplary embodiment of the present disclosure;

FIG. 4 is a top perspective view of a packaging kit in accordance with an exemplary embodiment of the present disclosure;

FIG. 5 is an exploded view of the interior components of the packaging kit of FIG. 4 in accordance with an exemplary embodiment of the present disclosure;

FIG. 6 is a flow chart illustrating a method for packaging a biological sample in accordance with an exemplary embodiment of the present disclosure; and

FIG. 7 is a flow chart illustrating a method for packaging a biological sample in accordance with an exemplary embodiment of the present disclosure.

### DETAILED DESCRIPTION

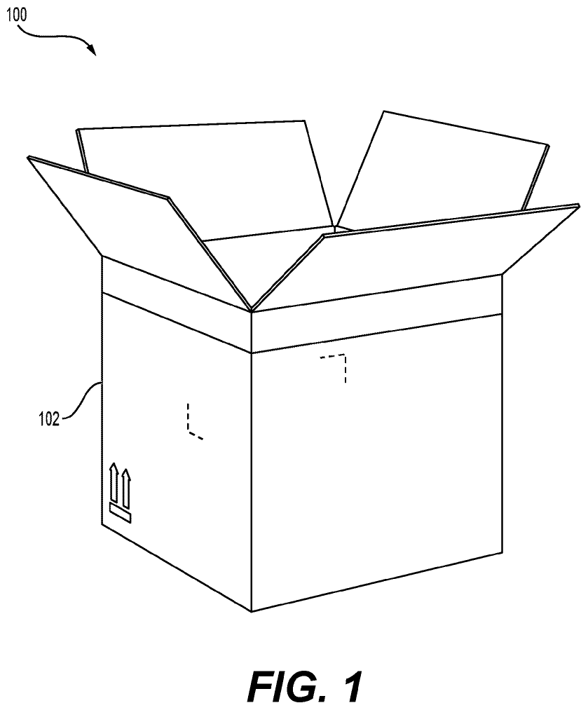

Exemplary embodiments of the present disclosure provide a packaging and transporting device for biological samples. An embodiment of the present disclosure is shown as an exemplary device in FIGS. 1-5. In use, packaging and transporting device (“device”) 100 may be used for the packaging and transporting (e.g., shipping) of biological samples. Device 100 may include outer shipping container 102, inner shipping container 106, and packaging kit 108 to package and transport a biological sample. In some embodiments, device 100 is used for the packaging and transporting of tumor tissues. For example, device 100 may provide packaging kit 108 for packaging and securing one or more biological samples for shipping or transporting. The biological samples may be tumor tissues taken from a patient. For example, a sample of tumor tissue removed from a patient may be placed within a tumor tissue container or sample container, such as a vial, for storing of the tumor tissue. In some embodiments, a sample of tumor tissue is removed from a cancer patient. The tumor tissue may be combined with a media/reagent to preserve the life of the tumor tissue and prevent contamination or degradation of the tumor tissue.

In some embodiments, the sample of tumor tissue is stored with a media/reagent within the sample container. The media may be a transport media, such as a hypothermic transport media configured to improve and extend the preservation of biological material (e.g., cells, tissues, organs). In some embodiments, the sample of tumor tissue is stored with HypoThermosol within the sample container. The sample container may then be sealed and secured using packaging kit 108.

In use, device 100 may be sent to a medical location (e.g., hospital, outpatient center, surgery center, physician's office, clinic, etc.). Tumor tissue may be extracted from a cancer patient at the medical location. The extracted tumor tissue may be packaged using device 100 and sent to a processing facility for processing (e.g., amplification of cells). For example, the tumor tissue may contain leukocytes (e.g., T lymphocytes or T-cells). The tumor tissue may be sent to the processing facility for amplification of the T-cells. The amplified T-cells may be sent back to the medical location and administered, e.g., by infusion, to the patient for treatment of cancer.

In practice, a medical location may be provided device 100 to assist them with the transportation of a biological sample to another location, such as another medical location or a processing facility. Device 100 may provide the medical location with the proper labels, monitors. sensors, packaging, and cooling means to protect and preserve the biological sample during transportation or shipping Device 100 may include outer shipping container 102, packaging kit 108, inner shipping container 106, and cooling engine 104. Cooling engine 104 may be disposed and housed within inner shipping container 106. In some embodiments, packaging kit 108, inner shipping container 106, and cooling engine 104 are disposed within outer shipping container 102 when device 100 is sent to a medical location for packaging of the biological sample. For example, cooling engine 104 may be disposed within inner shipping container 106, and inner shipping container 106 and packaging kit 108 may be disposed within outer shipping container 102 of device 100. Outer shipping container 102 may be sealed to secure packaging kit 108 and inner shipping container 106, which contains cooling engine 104, within outer shipping container 102. In some embodiments, multiple packaging kits 108, inner shipping containers 106 and/or cooling engines 104 are disposed within outer shipping container 102.

In some embodiments, outer shipping container 102 is sent to a medical location and includes inner shipping container 106, packaging kit 108, and cooling engine 104. The medical location may use packaging kit 108 to secure a biological sample and then place the packaging kit 108 containing the secured biological sample and cooling engine 104 within inner shipping container 106. In some embodiments, the medical location uses inner shipping container 106 to send the biological sample secured via packaging kit 108 and cooling engine 104 to a manufacturing facility. For example, inner shipping container 106 may be configured to receive cooling engine 104 and the biological sample secured via packaging kit 108 to safely and securely transport the secured biological sample to another medical location or processing facility. In some embodiments, the medical location may use outer shipping container 102 to send cooling engine 104 and the biological sample secured via packaging kit 108 to another medical location or processing facility.

In some embodiments, packaging kit 108 includes one or more components for sealing, securing, and monitoring the biological sample. Using packaging kit 108, the biological sample may be sealed and secured and placed within inner shipping container 106 with cooling engine 104. Cooling engine 104 may be configured to keep the biological sample within a specific temperature range. For example, cooling engine 104 may be configured to keep the biological sample at temperature of −80° C. to 10° C. to ensure the preservation of the biological sample during transportation and shipping of device 100. In some embodiments, cooling engine 104 is a container of dry ice or other cooling material configured to keep the biological sample (e.g., tumor tissue) at a temperature of −80° C. to 10° C.

Referring to FIG. 1, device 100 may include outer shipping container 102. Outer shipping container 102 may be sized and shaped to receive packaging kit 108, inner shipping container 106, and cooling engine 104. In some embodiments, outer shipping container 102 is configured to receive and secure packaging kit 108, inner shipping container 106, and cooling engine 104. For example, outer shipping container 102 may store and secure packaging kit 108, inner shipping container 106, and cooling engine 104 for shipping to a medical location. Outer shipping container 102 may be configured to house packaging kit 108, inner shipping container 106, and cooling engine 104 for transporting of packaging kit 108, inner shipping container 106, and cooling engine 104 to a medical location. Outer shipping container 102 may allow for packaging kit 108, inner shipping container 106, and cooling engine 104 to be stored within a single package during transportation. A medical location may receive outer shipping container 102, which includes packaging kit 108, inner shipping container 106, and cooling engine 104, to allow the medical location to secure a biological sample taken from a patient and safely transport the biological sample to another location, such as a processing facility. In some embodiments, the processing facility transports the processed biological sample (e.g., amplified T cells) using inner shipping container 106 back to the location (e.g., medical location) that device 100 came from.

In some embodiments, outer shipping container 102 may be configured for multiple uses. For example, outer shipping container 102 may be configured to be sent to a medical location and then may be used by the medical location to secure and transport cooling engine 104 and a biological sample secured using packaging kit 108 to another location, such as a processing facility. Outer shipping container 102 may be substantially rectangular shaped. In some embodiments, outer shipping container 102 may be circular, octagonal, or any other shape suitable. In some embodiments, outer shipping container 102 is comprised of cardboard. In some embodiments, outer shipping container 102 may be comprised of a polymer or metal.

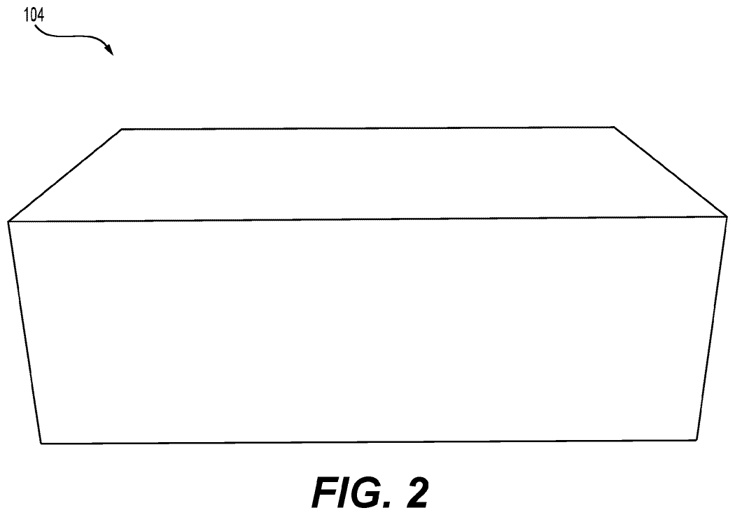

Referring to FIG. 2, device 100 may include a cooling pack or cooling engine 104. Cooling engine 104 may be configured to keep items stored within device 100 within a predetermined temperature range. Cooling engine 104 may be stored and housed within inner shipping container 106. For example, cooling engine 104 may remain within inner shipping container 106 when inner shipping container 106 is being transported within outer shipping container 102. In some embodiments, cooling engine 104 has an activated state and a deactivated state. In some embodiments, cooling engine 104 is removed from inner shipping container 106 only for the purpose of activating or deactivating cooling engine 104. Cooling engine 104 may be sent to a medical location in a deactivated state and may be activated by a user once the biological sample (e.g., tumor tissue sample) is placed within inner shipping container 106. For example, outer shipping container 102 may be sent to a medical location with packaging kit 108 and cooling engine 104 housed within inner shipping container 106 and in the deactivated state. A user may then remove cooling engine 104 from inner shipping container 106, activate cooling engine 104, place cooling engine 104 back into inner shipping container 106, label and secure the biological sample using packaging kit 108, and then may place the biological sample into inner shipping container 106 with cooling engine 104 in the activated state. In practice, device 100 may be sent to a medical location with cooling engine 104 in the deactivated state. Once the biological sample has been secured within a sample container using packaging kit 108, a user may activate cooling engine 104 and subsequently may place the sample container into inner shipping container 106 adjacent cooling engine 104. The user may then transport inner shipping container 106 to another location. In some embodiments, inner shipping container 106 is placed into outer shipping container 102, which is transported to another location. In some embodiments, cooling engine 104 is configured to keep a biological sample at a temperature of approximately 2° C. to approximately 8° C. In some embodiments, cooling engine 104 may be configured to keep the biological sample at a temperature of −80° C. to approximately 10° C., approximately −75° C. to approximately 0° C., or approximately −60° C. to approximately −10° C.

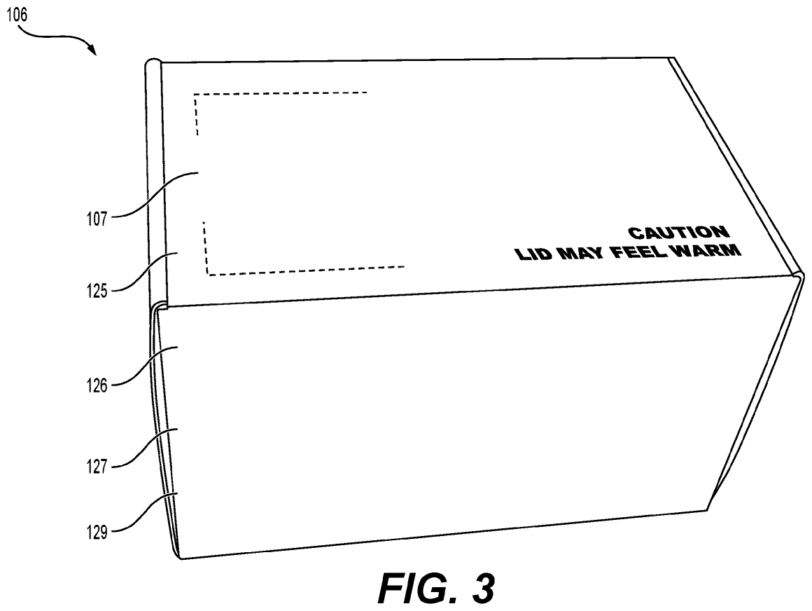

Referring to FIG. 3, device 100 may include inner shipping container 106. Inner shipping container 106 may be sized and shaped to receive cooling engine 104 and a biological sample secured via packaging kit 108. Inner shipping container 106 may also be sized and shaped to be disposed within outer shipping container 102. Inner shipping container 106 may be sent to a medical location to allow the medical location to send cooling engine 104 and the biological sample secured via packaging kit 108 to another medical location or processing facility. Inner shipping container 106 may be substantially rectangular shaped. In some embodiments, inner shipping container 106 may be circular, triangular, square, or any other shape suitable. Inner shipping container 106 may be comprised of a lightweight, yet strong material to allow for easy transportation of cooling engine 104 and a biological sample secured via packaging kit 108 while protecting the biological sample. In some embodiments, inner shipping container 106 is comprised of cardboard. In some embodiments, inner shipping container 106 may be comprised of a polymer, metal, or any other material suitable.

In some embodiments, inner shipping container 106 comprises area 107. Area 107 may be an area on an external surface of inner shipping container 106. Area 107 may be configured to receive a label. The label may be disposed within packaging kit 108 and may be retrieved from packaging kit 108 and placed on area 107. In some embodiments, a user may hand-write or print onto area 107. A user may use a label, hand-write, or print a destination for sending the biological sample secured via packaging kit 108. In some embodiments, inner shipping container 106 is configured to be folded. For example, inner shipping container 106 may be folded to substantially resemble a rectangular box. In some embodiments, inner shipping container 106 includes top panel 125 and extension portion 126 extending from top panel 125. Extension portion 126 may have flaps 127 that can be received within front wall 129. This allows inner shipping container 106 to be self-sealing without the need for tape or other sealing instruments. In some embodiments, inner shipping container 106 is comprised of a single piece that may be folded to resemble a rectangular box. In some embodiments, inner shipping container 106 and/or outer shipping container 102 include portions configured to receive individualized branding or other information.

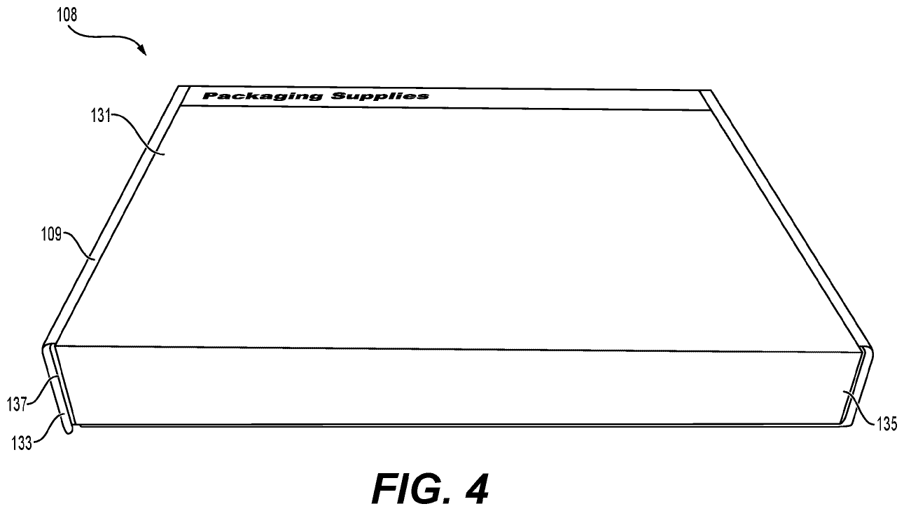

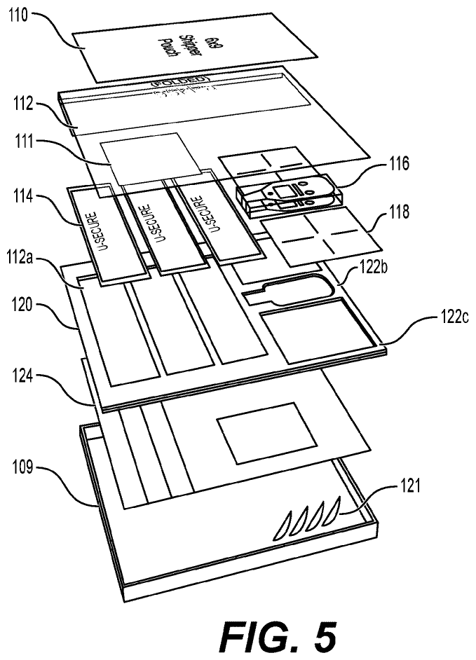

Referring to FIGS. 4-5, packaging kit 108 may include kit container 109. Kit container 109 may be configured to store and secure various components of packaging kit 108. For example, packaging kit 108 may include one or more labels, one or more securing features, one or more monitors, one or more sensors, and/or packaging, which may be stored within kit container 109. In some embodiments, kit container 109 is substantially rectangular shaped. In some embodiments, kit container 109 may be any shape suitable. Kit container 109 may be sized and shaped to store all the components of packaging kit 108 within and may be sized and shaped to fit within outer shipping container 102 and/or inner shipping container 106. For example, kit container 109 may be sized and shaped such that packaging kit 108 may be disposed within outer shipping container 102 and/or inner shipping container 106 during transportation of packaging kit 108 to/from the medical location.

In some embodiments, kit container 109 is configured to be folded. For example, kit container 109 may be folded to substantially resemble a thin rectangular box. Kit container 109 may have a thickness determined by the components disposed within packaging kit 108. For example, kit container 109 may have a thickness of approximately 1.5 inches. In some embodiments, kit container 109 may have thickness of approximately 0.5 inches to approximately 12 inches, approximately 1 inch to approximately 10 inches, approximately 2 inches to approximately 8 inches, approximately 3 inches to approximately 6 inches, greater than 12 inches, or less than 0.5 inches. In some embodiments, kit container 109 includes top panel 131 and extension portion 135 extending from top panel 131. Extension portion 135 may have flaps 133 that can be received within front wall 137. This allows kit container 109 to be self-sealing without the need for tape or other sealing instruments. In some embodiments, kit container 109 is comprised of a single piece that may be folded to resemble a rectangular box.

In some embodiments, kit container 109 includes slots 121. Slots 121 may be cut-outs within kit container 109 that are sized and shaped to receive and secure labels 124. For example, a portion (e.g., a corner) of label 124 may be inserted into slot 121 to secure label 124 in place during movement or transportation of packaging kit 108. In some embodiments, kit container 109 include a plurality of slots 121. For example, kit container 109 may include a number of slots 121 corresponding to the number of labels 124 such that each label 124 is secured into its respective slot 121. Slot 121 may be disposed in a corner of kit container 109 so that a corner of label 124 can be inserted into slot 121 to secure label 124 when it is disposed within kit container 109.

In some embodiments, packaging kit 108 includes one or more components for securing one or more biological samples for transportation. as shown in FIG. 5, packaging kit 108 may include one or more of: shipping pouch 110, transport bag 112, securing strip 114, sensor 116, securing element 118, insert 120, and label 124. In some embodiments, packaging kit 108 includes each component stacked together for easy storage and transportation. For example, during transporting of packaging kit 108, shipping pouch 110 may be disposed on transport bag 112, which may be disposed on insert 120, which may be disposed on label 124. Each component of packaging kit 108 may be layered together to reduce the overall size of packaging kit 108. In some embodiments, insert 120 may be disposed between label 124 and transport bag 112, and transport bag 112 may be disposed between shipping pouch 110 and insert 120.

In some embodiments, shipping pouch 110 is configured to receive one or more labels 124. For example, shipping pouch 110 may be configured to receive one or more labels 124, which may have destination information disposed on one or more labels 124. In some embodiments, packaging kit 108 includes two labels 124, one label 124 for the sample container and another label 124 for inner shipping container 106. Labels 124 may be patient-specific labels including information associated with a specific patient. In use, one or more labels 124 may be configured to include destination information and may be placed within shipping pouch 110, on inner shipping container 106 and/or on the sample container Shipping pouch 110 may be placed on inner shipping container 106. For example, shipping pouch 110 containing label 124 or shipping document (e.g., waybill) may be placed on area 107 of inner shipping container 106. Placing shipping pouch 110 with label 124 on area 107 allows for a courier to easily view the destination of inner shipping container 106. In some embodiments, shipping pouch 110 has a width of six inches and length of nine inches. In some embodiments, shipping pouch 110 may have a width of 1 inch to 12 inches, 3 inches to 9 inches, or 6 inches to 8 inches, and may have a length of 1 inch to 15 inches, 3 inches to 12 inches, or 6 inches to 9 inches.

In some embodiments, shipping pouch 110 is configured to receive a shipping document, such as a waybill. A user may insert a waybill into shipping pouch 110 and seal shipping pouch 110. The waybill may include destination information for the location and manner of transporting inner shipping container 106. In some embodiments, the shipping document (e.g., waybill) is printed by medical location. The medical location may pre-print a shipping document include non-patient specific information.

In some embodiments, insert 120 is comprised of foam. In some embodiments, insert 120 may be comprised of cardboard, polymer, or any other suitable type of protective material. Insert 120 may have a thickness of approximately 2 inches to approximately 0.1 inches, approximately 1.75 inches to approximately 0.5 inches, or approximately 0.75 inches to approximately 1 inch. In some embodiments, insert 120 has a thickness of approximately 0.75 inches.

In some embodiments, one or more securing strips 114, sensor 116, and one or more securing elements 118 are disposed within insert 120. For example, insert 120 may include cut outs 122a, 112b, and 122c sized and shaped to receive securing strip 114, sensor 116, and securing element 118, respectively In some embodiments, cut out 122a is sized and shaped to secure securing strip 114, cut out 122b is sized and shaped to secure sensor 116, and cut out 122c is sized and shaped to receive securing element 118. Cut outs 122a, 122b, and 122c are configured to secure strip 114, sensor 116, and securing element 118 to prevent damage to securing strip 114, sensor 116, and securing element 118 during transportation.

In some embodiments, insert 120 may include three cut outs 122a, one cut out 122b, and two cut outs 122c. In some embodiments, insert 120 may include any number of cut outs 122a, 122b, and 122c. For example, insert 120 may include a number of cut outs 122a, 122b, and 122c corresponding to the number of securing strip 114, sensor 116, and securing element 118. In some embodiments, cut out 122b is disposed between two cut outs 122c and one or more cut outs 122a are disposed below cut outs 122b and 122c. Cut out 122c may be configured to receive more than one securing element 118. In some embodiments, one or more cut outs 122c are each configured to receive four securing elements 118. In some embodiments, cut out 122c may be configured to receive any number of securing elements 118. In some embodiments, each cut out 122a is configured to receive one securing strip 114.

In some embodiments, one or more securing elements 118 are configured to ensure closure of the sample container receiving the biological sample. For example, as discussed above, the biological sample may be placed within a media (e.g., a transport media) within a sample container. The sample container may be secured with one or more securing elements 118 to prevent leakage or contamination of the biological sample within the sample container. In some embodiments, securing elements 118 are flexible films or adhesives or a self-sealing thermoplastic. For example, securing elements 118 may be parafilm, which may be secured around the sample container, such as adjacent the opening of the sample container once the biological sample and media have been placed in the sample container. In some embodiments, securing elements 118 may be tape, cellophane, Teflon, or any other suitable type of sealing element. In some embodiments, packaging kit 108 includes more than one securing element 118. For example, packaging kit 108 may include two, three, four, five, six, seven, eight, nine, ten, or greater than ten securing elements 118.

In some embodiments, transport bag 112 is configured to receive the biological sample and configured to be secured. For example, transport bag 112 may include an adhesive portion containing an adhesive such that transport bag 112 can be folded and the adhesive portion can be adhered to another portion of transport bag 112 thereby sealing transport bag 112. Transport bag 112 may include absorbent sheet 111. Absorbent sheet 111 may be configured to indicate that the sample container holding the biological sample has leaked or been damaged. Absorbent sheet 111 may be an absorbent pad configured to absorb liquid. In some embodiments, absorbent sheet 111 is configured to indicate when it has absorbed liquid. For example, absorbent sheet 111 may change colors when it has absorbed liquid thereby indicating that the sample container holding the biological sample has leaked or has been damaged during transportation. Once the sample container holding the biological sample has been placed within transport bag 112, transport bag 112 may be placed within inner shipping container 106 adjacent to cooling engine 104, which is also disposed within inner shipping container 106. Transport bag 112 may be 95 kPa liquid-tight transport bag. For example, transport bag 112 being a 95 kPa liquid-tight transport bag allows transport bag to withstand 95 kPa without leakage allowing the safe transportation of the sample container holding the biological sample. In some embodiments, transport bag 112 may be any type of bag configured to withstand pressure and prevent leakage.

In practice, the medical location receiving device 100 may place the sample container holding the biological sample into transport bag 112 having absorbent sheet 111. Transport bag 112 may be placed within inner shipping container 106 adjacent cooling engine 104. The medical location may transport or arrange for the transportation of inner shipping container 106, which houses transport bag 112 having the biological sample and cooling engine 104. The receiver (e.g., processing facility) of inner shipping container 106 may analyze absorbent sheet 111 to determine if the sample container holding the biological sample leaked or was damaged during transportation.

In some embodiments, packaging kit 108 includes securing strips 114. Securing strips 114 may be tamper evident strips and may be used secure inner shipping container 106. In some embodiments, inner shipping container 106 has an open position and a closed position. In the open position, the interior of inner shipping container 106 can be accessed. In the closed position, the interior of inner shipping container 106 cannot be accessed and the components stored within the interior are secure. Packaging kit 108 may include one or more securing strips 114 to secure inner shipping container 106 in the closed position.

Securing strips 114 may be placed on inner shipping container 106 in manner that prevents access to inner shipping container 106 and keeps inner shipping container 106 in the closed position. For example, once securing strips 114 have been applied to inner shipping container 106 when inner shipping container 106 is in the closed position, the interior of inner shipping container 106 may not be accessed without altering, damaging, or removing securing strips 114. In some embodiments, packaging kit 108 includes three securing strips 114 configured to be placed at different locations on inner shipping container 106 while inner shipping container 106 is in the closed position. Securing strips 114 may be placed on inner shipping container 106 such that once securing strips 114 have been placed on inner shipping container 106 when inner shipping container 106 is in the closed position, access to the interior of inner shipping container 106 is prevented. In some embodiments, securing strips 114 are used to prevent inner shipping container 106 from being placed in the open position without removing securing strips 114, thereby preventing access to the interior of inner shipping container 106.

Securing strips 114 may be anti-tampering strips or tape. For example, securing strips 114 may be difficult to remove once placed without causing visible damage to securing strips 114. In some embodiments, removing securing strips 114 results in visible damage to inner shipping container 106, thereby indicating to an individual that inner shipping container 106 has been tampered with or accessed. Removing securing strips 114 may result in leaving a residue or marking on inner shipping container 106 to indicate that inner shipping container 106 has been tampered with.

Packaging kit 108 may include one or more labels 124. Label 124 may be configured to include destination information, such as where the medical location intends to transport the biological sample such as to the processing facility. Label 124 may include de-identified information associated with patient that the biological sample was retrieved from. The de-identified patient information may be a number associated with the patient without revealing identifying information about the patient (e.g., name). Alternatively, label 124 may include identifying information about the patient (e.g., name, birthdate, age, etc.). In some embodiments, one label 124 is placed on the sample container containing the biological sample, and one label 124 is placed within shipping pouch 110 and placed on inner shipping container 106. In some embodiments, each label 124 includes different information. For example, one label 124 may include destination information affixed to inner shipping container 106 and another label 124 may include de-identified patient information to be placed on the sample container containing the biological sample. In some embodiments, each label 124 includes de-identified patient information and destination information. In other embodiments, each label 124 includes identifying information about the patient (e.g., name). In other embodiments, one label 124 includes destination information to be inserted into shipping pouch 110 placed on inner shipping container 106 and another label 124 includes identifying information about the patient (e.g., name) to be placed on the sample container containing the biological sample.

In some embodiments, labels 124 include adhesives to allow for easy affixing to inner shipping container 106. Labels 124 may be placed within shipping pouch 110 to protect labels 124 from damage. In some embodiments, packaging kit 108 includes more than one label 124. For example, packaging kit 108 may include two, three, four, five, six, seven, eight, nine, ten, or greater than ten labels 124.

In some embodiments, packaging kit 108 includes sensor 116. Sensor 116 may be a temperature sensor configured to monitor the temperature of inner shipping container 106 during transportation of the biological sample. In some embodiments, sensor 116 is configured to communicate with a remote server to notify an individual if the temperature inside inner shipping container 106 deviates from a predetermined range during transportation of the biological sample. In some embodiments, once activated, sensor 116 is configured to data log the temperatures within inner shipping container 106 Sensor 116 may be configured to transmit an alert or alarm when the temperature within inner shipping container 106 deviates from a predetermined temperature range. Sensor 116 may be configured to couple and/or communicate with an electronic device (e.g., USB, Bluetooth, Near Field Communication (NFC)). Sensor 116 may be configured to transmit temperatures detected during transportation of the biological sample such that a user may determine if the temperature deviated from the predetermined temperature range. Deviation from the predetermined temperature range may result in degradation of the biological sample.

In practice, a medical location may receive device 100, which may include inner shipping container 106, cooling engine 104, and packaging kit 108. Each of inner shipping container 106, cooling engine 104, and packaging kit 108 may be removed from outer shipping container 102. A biological sample may be retrieved from a patient and added to a transport media. The biological sample combined with the transport media may be stored within a sample container. Label 124 containing one or more of de-identified patient information and destination information may be added to the sample container. In some embodiments, label 124 containing identifying patient information (e.g., name, birthdate, age, etc.) and destination information are added to the sample container. Securing elements 118 from packaging kit 108 may be used to secure the sample container. Cooling engine 104 may be activated and placed within inner shipping container 106 along with sensor 116 from packaging kit 108. The secured sample container may be placed within transport bag 112. Transport bag 112 may then be placed within inner shipping container 106 adjacent activated cooling engine 104 and sensor 116. Inner shipping container 106 may be sealed and secured using securing strips 114. Another label 124 containing one or more of de-identified patient information and destination information may be inserted into shipping pouch 110, which may then be affixed to inner shipping container 106. In some embodiments, a label 124 containing one or more of patient identifying information (e.g., name, birthdate, age, etc.) and destination information are inserted into shipping pouch 110, which are then affixed to inner shipping container 106. Inner shipping container 106 containing the biological sample and cooling engine 104 may be transported to another location such as another medical location or processing facility.

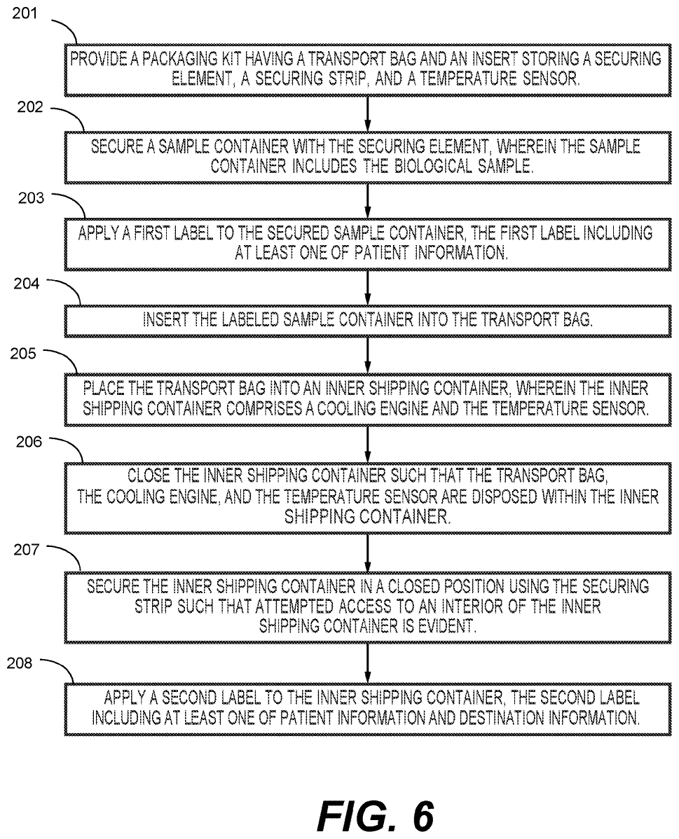

Referring to FIG. 6, in some embodiments, device 100 is used for a method for packaging a biological sample. In some embodiments, the method includes providing (201) device 100, including inner shipping container 106, cooling engine 104, and packaging kit 108. In some embodiments, packaging kit 108 may be provided and may include transport bag 112 and insert 120, which stores one or more securing elements 118, one or more securing strips 114, and sensor 116 (e.g., temperature sensor). In some embodiments, a sample container may be secured (202) with securing element 118 and the sample container may include a biological sample, which may be retrieved from a patient. In some embodiments, packaging kit 108 may include a plurality of labels 124. In some embodiments, the biological sample may be combined with a transport media and the transport media may be configured to prevent degradation of the biological sample.

In some embodiments, a first label of the plurality of labels 124 is applied (203) to the secured sample container. In some embodiments, the first label may include at least one of patient information and destination information. In some embodiments, the labeled sample container may be inserted (204) into transport bag 112 and transport bag 112 may be placed (205) into inner shipping container 106. In some embodiments, transport bag 112 may include absorbent sheet 111 configured to detect leakage of the labeled sample container within transport bag 112. In some embodiments, inner shipping container 106 may include cooling engine 104 and sensor 116, which may be a temperature sensor. In some embodiments, inner shipping container 106 is closed (206) such that transport bag 112, cooling engine 104, and sensor 116 are disposed within inner shipping container 106. In some embodiments, inner shipping container 106 may be secured (207) in a closed position using one or more securing strips 114 such that access to the interior of inner shipping container 106 is prevented. In some embodiments, one or more securing strips 114 are configured to leave a mark or residue on inner shipping container 106 if removed or altered. In some embodiments, a second label from the plurality of labels 124 is applied (208) to inner shipping container 106. In some embodiments, the second label may include patient information and destination information. In some embodiments, inner shipping container 106 may include the second label and is transported to a destination.

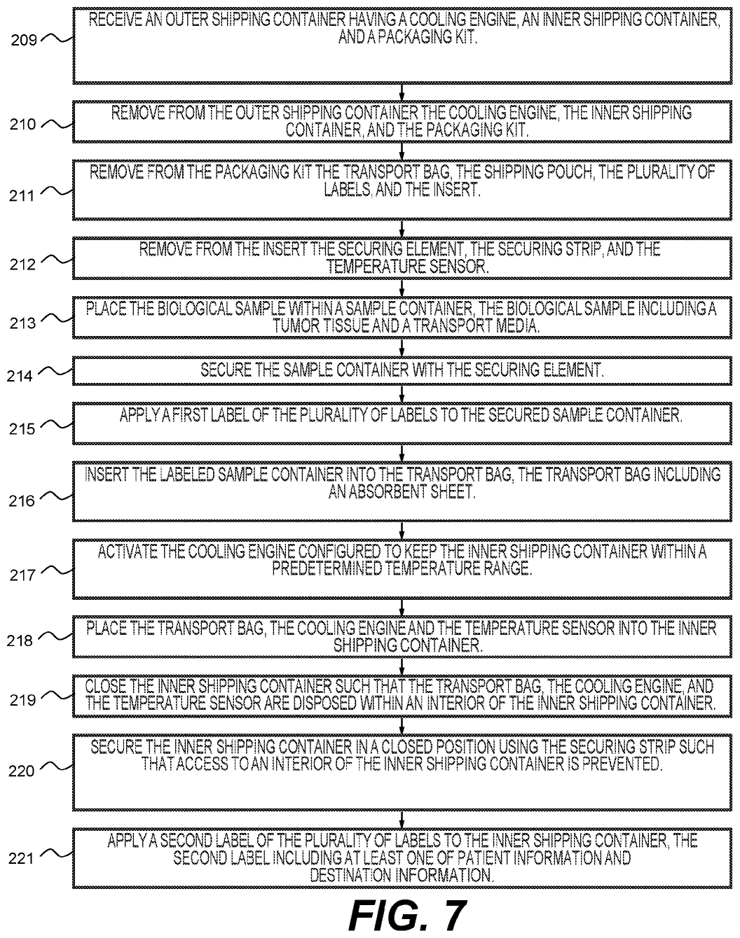

Referring to FIG. 7, in some embodiments, device 100 is used for a method for packaging a biological sample. In some embodiments, the method includes receiving (209) an outer shipping container 102 having a cooling engine 104, an inner shipping container 106, and a packaging kit 108. In some embodiments, the packaging kit 108 includes a transport bag 112, a shipping pouch 110, a plurality of labels 124 and an insert 120 storing a securing element 118, a securing strip 114, and a temperature sensor. In some embodiments, the cooling engine 104, the inner shipping container 106, and the packaging kit 108 is removed (210) from the outer shipping container 102. In some embodiments, the transport bag 112, the shipping pouch 110, the plurality of labels 124, and the insert 120, wherein the insert 120 includes cut outs sized and shaped to receive the securing element 118, the securing strip 114, and the temperature sensor are removed (211) from the packaging kit 108. In some embodiments, the securing element 118, the securing strip 114, and the temperature sensor are removed (212) from the insert 120. In some embodiments, the biological sample is placed (213) within a sample container, the biological sample including a tumor tissue and a transport media. In some embodiments, the sample container is secured (214) with the securing element 118. In some embodiments, a first label of the plurality of labels 124 is applied (215) to the secured sample container, the first label including at least one of patient information and destination information. In some embodiments, the labeled sample container is inserted (216) into the transport bag, the transport bag 112 including an absorbent sheet 111 configured to absorb leakage of the labeled sample container within the transport bag 112. In some embodiments, the cooling engine 104 is activated (217) such that the cooling engine 104 is configured to keep the inner shipping container 106 within a predetermined temperature range when the activated cooling engine 104 is disposed within the inner shipping container 106. In some embodiments, the transport bag 112, the cooling engine 104 and the temperature sensor are placed (218) into the inner shipping container 106. In some embodiments, the inner shipping container 106 is closed (219) such that the transport bag 112, the cooling engine 104, and the temperature sensor are disposed within an interior of the inner shipping container 106. In some embodiments, the inner shipping container 106 is secured (220) in a closed position using the securing strip 114 such that access to an interior of the inner shipping container 106 is prevented, wherein the securing strip 114 is configured to leave a mark or residue on the inner shipping container 106 if removed or altered. In some embodiments, a second label of the plurality of labels 124 is applied (221) to the inner shipping container 106, the second label including at least one of patient information and destination information.

In some embodiments, one or more securing strips 114 are removed from cut out 122a, sensor 116 is removed from cut out 122b, and one or more securing elements 118 are removed from cut out 122c. In some embodiments, cooling engine 104 may be activated such that cooling engine 104 is configured to keep inner shipping container 106 within a predetermined temperature range when cooling engine 104 is activated and disposed within inner shipping container 106. In some embodiments, the predetermined temperature range may be 0° C. to 10° C.

In some embodiments, outer shipping container 102 is received by a user and includes cooling engine 104, inner shipping container 106, and packaging kit 108. In some embodiments, a user may remove cooling engine 104, inner shipping container 106, and packaging kit 108 from outer shipping container 102 and may remove transport bag 112, shipping pouch 110, plurality of labels 124, and insert 120 from packaging kit 108.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

1. A packaging and transporting device for a biological sample, the packaging and transporting device comprising:
an insert;
a securing strip;
a securing element;
a temperature sensor, wherein the insert comprises a plurality of cut outs sized and shaped to receive the securing strip, the securing element, and the temperature sensor;
a plurality of labels, wherein at least one of the plurality of labels includes patient information;
a shipping pouch sized and shaped to receive a shipping document; and
a transport bag configured to receive a sample container.

2. The packaging and transporting device of claim 1, wherein the plurality of cut outs includes a first cut out, a second cut out, and a third cut out.

3. The packaging and transporting device of claim 2, wherein the first cut out of the insert is configured to receive and store the securing strip, the second cut out of the insert is configured to receive and store the temperature sensor, and the third cut out of the insert is configured to receive and store the securing element.

4. The packaging and transporting device of claim 1, wherein the insert is disposed between the transport bag and the plurality of labels.

5. The packaging and transporting device of claim 1, wherein the insert is disposed between the transport bag and the plurality of labels, and the transport bag is disposed between the shipping pouch and the insert.

6. The packaging and transporting device of claim 1, further comprising:
an inner shipping container sized and shaped to receive the transport bag and a cooling engine, wherein the inner shipping container is smaller in size than the packaging and transporting device.

7. The packaging and transporting device of claim 6, wherein the plurality of labels includes a first label configured to be applied to the sample container and a second label configured to be applied to the inner shipping container.

8. The packaging and transporting device of claim 6, wherein the securing strip is configured to leave a mark on the inner shipping container if removed or altered.

9. The packaging and transporting device of claim 1, wherein the securing strip is disposed within one of the plurality of cut outs of the insert.

10. The packaging and transporting device of claim 1, wherein the insert includes three or more securing strips.

11. The packaging and transporting device of claim 10, wherein the three or more securing strips are disposed within the plurality of cut outs of the insert.

12. The packaging and transporting device of claim 1, wherein the insert includes four or more securing elements.

13. The packaging and transporting device of claim 12, wherein the four or more securing elements are disposed within the plurality of cut outs of the insert.

14. The packaging and transporting device of claim 1, wherein the patient information comprises identifying patient information.

15. The packaging and transporting device of claim 14, wherein the identifying patient information comprises one or more of a patient's name, birthdate, age, etc.

16. The packaging and transporting device of claim 1, further comprising:
a cooling engine configured to maintain a temperature range of approximately −80° C. to approximately 10° C. upon activation of the cooling engine.

17. The packaging and transporting device of claim 1, wherein the transport bag includes an absorbent sheet configured to absorb leakage of the sample container.

18. The packaging and transporting device of claim 1, wherein the packaging and transporting device includes two or more labels.

19. The packaging and transporting device of claim 1, wherein the temperature sensor is disposed within one of the plurality of cut outs of the insert.

20. The packaging and transporting device of claim 1, wherein the transport bag is configured to withstand up to 95 kPa without leaking.

21. The packaging and transporting device of claim 1, wherein the patient information comprises de-identified patient information.

22. The packaging and transporting device of claim 1, wherein the insert is disposed between the plurality of labels and one of the shipping pouch and the transport bag.

23. The packaging and transporting device of claim 1, further comprising:
kit container housing the insert, the plurality of labels, the shipping pouch, and the transport bag.

24. A packaging and transporting device for a biological sample, the device comprising:
a packaging kit comprising:
an insert having a securing strip, a securing element, and a temperature sensor, wherein the insert includes a plurality of cut outs sized and shaped to receive the securing strip, the securing element, and the temperature sensor;
a plurality of labels including one or more of patient information and destination information;
a transport bag configured to receive a sample container containing the biological sample;
a shipping pouch;
a cooling engine configured to maintain a temperature range of approximately 0° C. to approximately 10° C.;
an inner shipping container; and
an outer shipping container sized and shaped to receive the inner shipping container, the cooling engine, and the packaging kit,
wherein the inner shipping container includes an area to receive at least one of the plurality of labels and the inner shipping container is sized and shaped to receive the cooling engine and the transport bag.

25. The packaging and transporting device of claim 24, wherein the insert is disposed between the transport bag and the plurality of labels.

26. The packaging and transporting device of claim 24, wherein the transport bag is disposed between the shipping pouch and the insert.

27. A method for packaging a biological sample, the method comprising:
providing a packaging kit having a transport bag and an insert storing a securing element, a securing strip, and a temperature sensor;
securing a sample container with the securing element, wherein the sample container includes the biological sample;
applying a first label to the secured sample container, the first label including at least one of patient information;
inserting the labeled sample container into the transport bag;
placing the transport bag into an inner shipping container, wherein the inner shipping container comprises a cooling engine and the temperature sensor;
closing the inner shipping container such that the transport bag, the cooling engine, and the temperature sensor are disposed within the inner shipping container;
securing the inner shipping container in a closed position using the securing strip such that attempted access to an interior of the inner shipping container is evident; and
applying a second label to the inner shipping container, the second label including at least one of patient information and destination information.

28. The method of claim 27, wherein the packaging kit further includes a shipping pouch and a plurality of labels including the first label and the second label.

29. The method of claim 28, wherein, within the packaging kit, the insert is disposed between the transport bag and the plurality of labels.

30. The method of claim 28, wherein, within the packaging kit, the transport bag is disposed between the shipping pouch and the insert.

31. The method of claim 27 further comprising:
removing the securing strip from a first cut out of the insert;
removing the temperature sensor from a second cut out of the insert; and
removing the securing element from a third cut out of the insert.

32. The method of claim 31, wherein the first cut out of the insert is configured to receive and store the securing strip.

33. The method of claim 31, wherein the second cut out of the insert is configured to receive and store the temperature sensor.

34. The method of claim 31, wherein the third cut out of the insert is configured to receive and store the securing element.

35. The method of claim 27, further comprising:
activating the cooling engine such that the cooling engine is configured to keep the inner shipping container within a predetermined temperature range when the activated cooling engine is disposed within the inner shipping container.

36. The method of claim 35, wherein the predetermined temperature range is 0° C. to 10° C.

37. The method of claim 27, further comprising:
transporting the inner shipping container having the second label to a destination.

38. The method of claim 27 further comprising:
retrieving the biological sample from a patient.

39. The method of claim 27, further comprising:
combining the biological sample with a transport media, wherein the transport media is configured to prevent degradation of the biological sample.

40. The method of claim 27, wherein the biological sample comprises a plurality of tumor tissue retrieved from a patient.

41. The method of claim 27, wherein the transport bag includes an absorbent sheet configured to absorb leakage of the sample container.

42. The method of claim 27, wherein the securing strip is configured to leave a mark on the inner shipping container if removed or altered.

43. The method of claim 27, wherein the packaging kit includes three or more securing strips.

44. The method of claim 27, wherein the packaging kit includes four or more securing elements.

45. The method of claim 27, wherein the packaging kit includes two or more labels.

46. The method of claim 27, wherein the transport bag is configured to withstand up to 95 kPa without leaking.

47. The method of claim 27, wherein the patient information comprises de-identified patient information.

48. The method of claim 27, wherein the patient information comprises identifying patient information.

49. The method of claim 48, wherein the patient information comprises a patient's name, birthdate, and/or age.

50. The method of claim 27, wherein the packaging kit includes a kit container, the kit container configured to receive and store the transport bag and the insert.

51. A method for packaging a biological sample, the method comprising
receiving an outer shipping container having a cooling engine, an inner shipping container, and a packaging kit, wherein the packaging kit having a transport bag, a shipping pouch, a plurality of labels and an insert storing a securing element, a securing strip, and a temperature sensor;
removing from the outer shipping container the cooling engine, the inner shipping container, and the packaging kit;
removing from the packaging kit the transport bag, the shipping pouch, the plurality of labels, and the insert, wherein the insert includes cut outs sized and shaped to receive the securing element, the securing strip, and the temperature sensor;
removing from the insert the securing element, the securing strip, and the temperature sensor;
placing the biological sample within a sample container, the biological sample including a tumor tissue and a transport media;
securing the sample container with the securing element;
applying a first label of the plurality of labels to the secured sample container, the first label including at least one of patient information and destination information;
inserting the labeled sample container into the transport bag, the transport bag including an absorbent sheet configured to absorb leakage of the labeled sample container within the transport bag;
activating the cooling engine such that the cooling engine is configured to keep the inner shipping container within a predetermined temperature range when the activated cooling engine is disposed within the inner shipping container;
placing the transport bag, the cooling engine and the temperature sensor into the inner shipping container;
closing the inner shipping container such that the transport bag, the cooling engine, and the temperature sensor are disposed within an interior of the inner shipping container;
securing the inner shipping container in a closed position using the securing strip such that access to an interior of the inner shipping container is prevented, wherein the securing strip is configured to leave a mark or residue on the inner shipping container if removed or altered; and
applying a second label of the plurality of labels to the inner shipping container, the second label including at least one of patient information and destination information.
